(19) United States
(12) Patent Application Publication
(10) Pub. No.: US 2025/0248971 A1
(43) Pub. Date: Aug. 7, 2025

(54) PHARMACEUTICAL COMPOSITION AND METHOD USING THE SAME

(71) Applicant: Anxo Pharmaceutical Co., Ltd., Taipei City (TW)

(72) Inventors: Chia-Yu Su, Taipei City (TW); Hsiu-Chuan Tseng, Taipei City (TW); Hua-Jing Jhan, Taipei City (TW); Tse-Hsien Chen, Taipei City (TW); Chi-Heng Jian, Taipei City (TW)

(73) Assignee: Anxo Pharmaceutical Co., Ltd., Taipei City (TW)

(21) Appl. No.: 19/043,532

(22) Filed: Feb. 3, 2025

Related U.S. Application Data
(60) Provisional application No. 63/549,583, filed on Feb. 5, 2024.

(51) Int. Cl. A61K 31/404; A61K 47/14; A61K 47/34; A61K 47/44

(52) U.S. Cl. CPC A61K 31/404; A61K 47/14; A61K 47/34; A61K 47/44

(57) ABSTRACT

Provided is a pharmaceutical composition includes Nintedanib or a pharmaceutically acceptable salt thereof and a stabilizer. The stabilizer includes polyoxylglyceride. Also provided is a method for preventing, ameliorating, or treating fibrotic disease and/or lung cancer. The method includes administering an effective amount of the pharmaceutical composition to a subject in need thereof.

### CROSS REFERENCE TO RELATED APPLICATIONS

This application claims the benefit of U.S. Provisional Application No. 63/549,583, filed on Feb. 5, 2024. The content of the application is incorporated herein by reference.

### BACKGROUND OF THE INVENTION

### 1. Field of the Invention

The present disclosure relates to a pharmaceutical composition, and more particularly to a pharmaceutical composition for preventing, ameliorating, or treating fibrotic disease and/or lung cancer.

### 2. Description of the Prior Art

Idiopathic pulmonary fibrosis (IPF) is a chronic and progressive fibrosing interstitial pneumonia that primarily affects the elderly and is limited to the lungs. The pathogenic mechanisms of IPF remain unclear. Potential risk factors include smoking, viral infections, environmental pollution, genetics, and medications. Current studies indicate that the pathogenesis of IPF may involve repeated injury of alveolar epithelial cells, leading to tissue fibrosis. Damaged epithelial cells secrete a large amount of growth factors, thereby activating fibroblasts, and promoting the activation, proliferation, and differentiation of the fibroblasts. Therefore, fibroblastic foci including the activated fibroblasts and myofibroblasts are developed.

Antifibrotic medications including Nintedanib was developed, which blocks intracellular signaling, inhibits proliferation, migration, and transformation of the fibroblasts, and reduces the fibrotic process and the rate of decline in lung function.

Ofev® (active substance: Nintedanib), developed by Boehringer Ingelheim, is a soft capsule recommended to be taken twice a day after meal. However, according to the Japanese Interview Form (IF), Ofev® does not meet the required dissolution rate in accelerated stability test. If the dissolution rate of a pharmaceutical product fails to meet the requirement of the accelerated stability test, intermediate and long-term stability data must be provided to prove the pharmaceutical product's shelf life. Furthermore, previous studies reported that Ofev® must be stored below 25° C., thereby increasing the cost of the transportation and storage, especially in tropical regions such as Southeast Asia.

It is known, U.S. Pat. No. 9,907,756B2 discloses a soft capsule containing Nintedanib. However, U.S. Pat. No. 9,907,756B2 fails to disclose how to maintain or improve the stability and dissolution of the soft capsule for a long time.

Therefore, there is still an unmet need in the art to overcome a disadvantage of the unstable dissolution of the pharmaceutical product containing Nintedanib in the accelerated stability test.

### SUMMARY OF THE INVENTION

In view of the foregoing, the present disclosure provides a pharmaceutical composition. The pharmaceutical composition includes Nintedanib or a pharmaceutically acceptable salt thereof, and a stabilizer containing polyoxylglyceride.

The present disclosure also provides a method for preventing, ameliorating, or treating fibrotic disease and/or lung cancer. The method includes administering an effective amount of the pharmaceutical composition of the present disclosure to a subject in need thereof.

In at least one embodiment of the present disclosure, the stabilizer containing polyoxylglyceride may bring about a superior effect on increasing the stability of the pharmaceutical composition for a longer period of time and improving the dissolution thereof as compared with the commercially available drugs.

The objectives of the present disclosure will no doubt become understandable to those of ordinary skill in the art after reading the following detailed description of the embodiments that is illustrated in the various figures and drawings.

### BRIEF DESCRIPTION OF THE DRAWINGS

FIG. 1 illustrates the dissolution profile of the comparative group (Ofev®) and experimental group 1 according to an embodiment of the present disclosure.

FIG. 2 illustrates the dissolution profile of the comparative group (Ofev®) and experimental group 2 according to an embodiment of the present disclosure.

### DETAILED DESCRIPTION

The following embodiments are provided to illustrate the present disclosure in detail. A person having ordinary skills in the art can easily understand the advantages and effects of the present disclosure after reading the disclosure of this specification, and also can implement or apply in other different embodiments. Therefore, it is possible to modify and/or alter the following embodiments for carrying out this disclosure without contravening its scope for different aspects and applications, and any element or method within the scope of the present disclosure disclosed herein can combine with any other element or method disclosed in any embodiments of the present disclosure.

In order that the present disclosure may be more readily understood, certain terms are first defined. In addition, it should be noted that whenever a value or range of values of a parameter are recited, it is intended that values and ranges intermediate to the recited values are also intended to be part of the present disclosure.

As used herein, the singular forms “a,” “an,” and “the” include plural referents, unless expressly and unequivocally limited to one referent. For example, “an active ingredient” means one active ingredient or more than one active ingredient, e.g., a plurality of active ingredients. The term “or” is used interchangeably with the term “and/or” unless the context clearly indicates otherwise.

As used herein, the term “comprising,” “comprises” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, parts, steps, or connections, etc., and should not exclude other limitations.

As used herein, the phrase “at least one,” in reference to a list of one or more elements, should be understood to mean at least one element selected from any one or more of the elements in the list of elements, but not necessarily including at least one of each element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

The numeral ranges used herein are inclusive and combinable, any numeral value that falls within the numeral scope herein could be taken as a maximum or minimum value to derive the sub-ranges therefrom. For example, the numeral range “0.5% to 5%” includes any sub-ranges between the minimum value of 0.5% to the maximum value of 5%, such as the sub-ranges from 0.5% to 3%, from 0.7% to 5%, from 3% to 4% and so on. In addition, a plurality of numeral values used herein can be optionally selected as maximum and minimum values to derive numerical ranges. For instance, the numerical ranges of 0.5% to 5%, 0.7% to 5%, and 0.5% to 0.7% can be derived from the numeral values of 0.5%, 0.7%, and 5%.

The term “subject” refers to human or other animals. In at least one embodiment of the present disclosure, the subject may be a mammal, such as, but not limited to a human, non-human primate, canine, feline, murine, bovine, equine, porcine, sheep, deer, wolf, fox, and rabbit.

As used herein, the term “carrier” refers to a pharmaceutically acceptable material, vehicle, or composition, such as a solid or liquid filler, binder, diluent, preservative, biocompatible solvent, disintegrating agent, lubricant, suspending agent, flavoring agent, encapsulating material, thickening agent/thickener, acid, surfactant, complexation agent, wetting agent, or any combination thereof. In some embodiments, each component is “pharmaceutically acceptable” in the sense of being compatible with the other ingredients of a pharmaceutical formulation, and suitable for use in contact with the organ or tissue of a subject without excessive toxicity, allergic response, irritation, immunogenicity, or other complications or problems. See, e.g., Remington: The Science and Practice of Pharmacy, 22nd ed.; Allen Ed.: Philadelphia, PA, 2012; Handbook of Pharmaceutical Excipients, 7th ed.; Rowe et al., Eds.; The Pharmaceutical Press and the American Pharmaceutical Association: 2012; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

As used herein, the term “effective amount” refers to the amount of an active agent or a pharmaceutical composition that is sufficient to bring about an effect on treating, preventing, or ameliorating a disorder, disease, or condition of a subject in need thereof. The effective amount may vary by a person ordinarily skilled in the art, depending on excipient usage, routes of administration, the possibility of co-usage with other therapeutic treatment, or the condition to be treated, but the present disclosure is not limited thereto.

As used herein, the term “administer,” “administering” or “administration” refer to the placement of an active ingredient into a subject by a method or route which results in at least partial localization of the active ingredient at a desired site to produce the desired effect. For example, the active ingredient of the present disclosure may be administered to a subject by oral administration, injection, subcutaneous administration, intramuscular administration, topical administration, or nasal administration, but the present disclosure is not limited thereto.

As used herein, the term “treat,” “treating,” or “treatment” refers to obtaining a desired pharmacologic and/or physiologic effect. The effect may be prophylactic in terms of completely or partially preventing a disease or symptoms or conditions thereof or may be therapeutic in terms of completely or partially curing, alleviating, relieving, remedying, or ameliorating a disease or an adverse effect attributable to the disease or symptoms or conditions thereof.

As used herein, the term “prevent,” “preventing,” or “prevention” does not require that the disease state be completely thwarted. Rather, as used herein, the term “prevent,” “preventing,” or “prevention” refers to the ability of the skilled artisan to identify a population that is susceptible to disorders, such that administration of the extracellular vesicles of the present disclosure may occur prior to onset of a disease. The term does not imply that the disease state is completely avoided.

The terms “ameliorate” and “ameliorating” refer to easing or reducing one or more symptoms (e.g., pain) of a disorder, disease, or condition. The terms can also refer to reducing adverse effects associated with an active ingredient. In some embodiments, the beneficial effects that a subject derives from a prophylactic or therapeutic agent do not result in a cure of the disorder, disease, or condition.

In at least one embodiment of the present disclosure, the stabilizer may contain polyoxylglyceride including at least one selected from the group consisting of lauroyl polyoxylglyceride, caprylocaproyl polyoxylglyceride, stearoyl polyoxylglyceride, and linoleoyl polyoxylglyceride, but the present disclosure is not limited thereto. In some embodiments, the lauroyl polyoxylglyceride may be lauroyl polyoxyl-32 glycerides. In some embodiments, the caprylocaproyl polyoxylglyceride may be caprylocaproyl polyoxyl-8 glycerides. In some embodiments, the stearoyl polyoxylglyceride may be stearoyl polyoxyl-32 glyceride. In some embodiments, the linoleoyl polyoxylglyceride may be linoleoyl polyoxyl-6 glyceride.

In at least one embodiment of the present disclosure, the stabilizer may be in an amount of 0.5% to 5.0% by weight of the pharmaceutical composition, such as, but not limited to 0.5% to 4.0%, 0.9% to 3.0%, 0.9% to 2%, or 1.3% to 2.0% by weight of the pharmaceutical composition. In some embodiments, the stabilizer may be in an amount of 0.5%, 0.6%, 0.7%, 0.8%, 0.9%, 1.0%, 1.5%, 2%, 2.5%, 3.0%, 3.5%, 4.0%, 4.5%, or 5.0% by weight of the pharmaceutical composition, but the present disclosure is not limited thereto.

In at least one embodiment of the present disclosure, the stabilizer may have a hydrophilic-lipophilic balance (HLB) value of 11.0 to 14.0, such as, but not limited to 11.0 to 13.5, 11.5 to 13.0, or 12 to 13.5. In some embodiments, the stabilizer may have a HLB value of 11, 11.5, 12, 12.5, 13, 13.5, or 14, but the present disclosure is not limited thereto.

In at least one embodiment of the present disclosure, the pharmaceutically acceptable salt of the Nintedanib may be at least one selected from the group consisting of Nintedanib methanesulfonate, Nintedanib esylate, Nintedanib ethanedisulfonate, Nintedanib isethionate, Nintedanib tosylate, Nintedanib sulfate, Nintedanib phosphate, Nintedanib benzenesulfonate, Nintedanib camphorsulfonate, Nintedanib citrate, Nintedanib bromide, Nintedanib chloride, Nintedanib benzoate, Nintedanib saccharinate, Nintedanib L-lactate, Nintedanib L-malate, Nintedanib D-malate, Nintedanib gentisate, Nintedanib glycolate, Nintedanib naphthalene-1,5-disulfonate, Nintedanib L-tartrate, Nintedanib D-tartrate, Nintedanib glycinate, Nintedanib malonate, Nintedanib oxalate, Nintedanib camphorate, Nintedanib mandelate, Nintedanib salicylate, Nintedanib ascorbate, and Nintedanib xinafoate, but the present disclosure is not limited thereto. In some embodiments, the pharmaceutically acceptable salt may be Nintedanib esylate.

In at least one embodiment of the present disclosure, the Nintedanib or the pharmaceutically acceptable salt thereof may be in an amount of 20% to 50% by weight of the pharmaceutical composition, such as, but not limited to 20% to 45%, 30% to 45%, or 35% to 50% by weight of the pharmaceutical composition. In some embodiments, the Nintedanib or the pharmaceutically acceptable salt thereof may be in an amount of 20%, 25%, 30%, 35%, 40%, 45%, or 50% by weight of the pharmaceutical composition, but the present disclosure is not limited thereto.

In at least one embodiment of the present disclosure, the pharmaceutical composition may further include a carrier and/or a surfactant.

In at least one embodiment of the present disclosure, the carrier may include a lipophilic substance. In some embodiments, the lipophilic substance may be at least one selected from the group consisting of vegetable oil, medium chain triglyceride, medium chain partial glyceride, animal derived oil, sorbitan monostearate, ethyl oleate, corn oil glyceride, acetylated monoglyceride, caprylic/capric triglyceride, caprylic/capric/succinic triglyceride, caprylic/capric/linoleic triglyceride, oleic acid, glycerol monoleate, macrogolglycerol caprylocaprate, propylene glycol dicaprylate, and glycerol monolinolate. In some embodiments, the lipophilic substance may be the vegetable oil, the medium chain triglyceride, or a combination thereof; and wherein the vegetable oil is soybean oil, coconut oil, castor oil, or a combination thereof.

In at least one embodiment of the present disclosure, the carrier has a hydrophilic-lipophilic balance (HLB) value of 1 to 3.

In at least one embodiment of the present disclosure, the carrier may be in an amount of 30% to 70% by weight of the pharmaceutical composition, such as, but not limited to 30% to 60%, 35% to 55%, or 40% to 60% by weight of the pharmaceutical composition. In some embodiments, the carrier may be in an amount of 30%, 35%, 40%, 45%, 50%, 55%, 60%, 65%, or 70% by weight of the pharmaceutical composition, but the present disclosure is not limited thereto.

In at least one embodiment of the present disclosure, the surfactant may include at least one selected from the group consisting of propylene glycol monolaurate, polyglycerol-3 dioleate, propylene glycol monocaprylate, propylene glycol dicaprylate, lecithin, polyethylene glycol sorbitan monolaurate, polyethylene glycol sorbitan monooleate, macrogolglycerol hydroxystearate, macrogolglycerol ricinoleate, macrogolglycerol linoleate, polyglycerol-6 dioleate, and D-a-tocopherol polyethylene glycol succinate, but the present disclosure is not limited thereto. In some embodiments, the surfactant may include propylene glycol monolaurate, lecithin, or a combination thereof.

In at least one embodiment of the present disclosure, the surfactant may have a HLB value of 3 to 6, such as, but not limited to 3 to 5, 4 to 6, or 4 to 5. In some embodiments, the surfactant may have a HLB value of 3.0, 3.5, 4.0, 4.5, 5.0, 5.5, or 6.0, but the present disclosure is not limited thereto.

In at least one embodiment of the present disclosure, the surfactant may be in an amount of 0.3% to 25.0% by weight of the pharmaceutical composition, such as, but not limited to 0.3% to 20.0%, 5.0% to 15.0%, or 10.0% to 25.0% by weight of the pharmaceutical composition. In some embodiments, the surfactant may be in an amount of 0.3%, 0.4%, 0.5%, 0.6%, 0.7%, 0.8%, 0.9%, 1.0%, 5.0%, 10.0%, 15.0%, 20.0%, or 25.0% by weight of the pharmaceutical composition, but the present disclosure is not limited thereto.

In at least one embodiment of the present disclosure, the pharmaceutical composition may include the Nintedanib or the pharmaceutically acceptable salt thereof in an amount of 20% to 69.2% by weight of the pharmaceutical composition; the stabilizer in an amount of 0.5% to 5% by weight of the pharmaceutical composition; the carrier in an amount of 30% to 55% by weight of the pharmaceutical composition; and the surfactant in an amount of 0.3% to 20% by weight of the pharmaceutical composition. In some embodiments, the pharmaceutical composition may include the Nintedanib or the pharmaceutically acceptable salt thereof is in an amount of 20% to 59.2% by weight of the pharmaceutical composition; the stabilizer is in an amount of 0.5% to 5% by weight of the pharmaceutical composition; the carrier is in an amount of 40% to 50% by weight of the pharmaceutical composition; and the surfactant is in an amount of 0.3% to 15% by weight of the pharmaceutical composition.

In at least one embodiment of the present disclosure, the stabilizer may have a HLB value of 11 to 14; the carrier may have a HLB value of 1 to 3; and the surfactant may have a HLB value of 3 to 6.

At least one embodiment of the present disclosure may provide a capsule including the pharmaceutical composition of the present disclosure. In some embodiments, the capsule may be a soft capsule or a hard capsule. In some embodiments, the soft capsule or hard capsule may include a capsule shell encapsulating the pharmaceutical composition of the present disclosure. In some embodiments, the capsule shell may include gelatin, solvents, emollients, opacifiers, plasticizers, sweetening agents, dyes, pigments, flavoring agents, sugar, oligosaccharides or polysaccharides, adjuvants, preservatives and/or one or more active ingredients, but the present disclosure is not limited thereto. In some embodiments, the soft capsule may have a capsule shell including Gelatin 160, glycerin, titanium dioxide, iron oxide red, iron oxide yellow, and/or purified water.

At least one embodiment of the present disclosure may provide a tablet including the pharmaceutical composition of the present disclosure. In some embodiments, the tablet may be a chewable tablet, a film-coated tablet, an orally disintegrating tablet, or an enteric-coated tablet. In some embodiments, the tablet may include a tablet shell encapsulating the pharmaceutical composition of the present disclosure.

In some embodiments, the pharmaceutical composition of the present disclosure may be, but is not limited to, a formulation to injection, dry powder, a tablet, an oral liquid, a wafer, a film, a lozenge, a capsule, a granule, or a pill.

At least one embodiment of the present disclosure provides a use of Nintedanib or a pharmaceutically acceptable salt thereof for manufacture of a pharmaceutical composition for preventing, ameliorating, or treating fibrotic disease and/or lung cancer. The pharmaceutical composition includes a stabilizer containing polyoxylglyceride.

At least one embodiment of the present disclosure provides a pharmaceutical composition for use in preventing, ameliorating, or treating fibrotic disease and/or lung cancer. The pharmaceutical composition includes Nintedanib or a pharmaceutically acceptable salt thereof, and a stabilizer including polyoxylglyceride.

In at least one embodiment of the present disclosure, the fibrotic disease may be idiopathic pulmonary fibrosis, and the lung cancer may be non-small cell lung cancer.

In some embodiments, the fibrotic disease may be, but is not limited to, fibrosis and remodeling of lung tissue in chronic obstructive pulmonary disease, fibrosis and remodeling of lung tissue in chronic bronchitis, fibrosis and remodeling of lung tissue in emphysema, lung fibrosis and pulmonary diseases with a fibrotic component, fibrosis and remodeling in asthma, fibrosis in rheumatoid arthritis, virally induced hepatic cirrhosis, radiation-induced fibrosis, post angioplasty restenosis, chronic glomerulonephritis, renal fibrosis, diseases of the skin with a fibrotic component, and/or excessive scarring.

In some embodiments, the lung cancer may be, but is not limited to, a non-small cell lung cancer, a pulmonary squamous cell carcinoma, a small cell lung cancer, and/or a lung adenocarcinoma.

In at least one embodiment of the present disclosure, the operation process of the dissolution test may include: using a paddle method; a speed of 70 rpm to 150 rpm; and a dissolution medium of 700 ml to 1000 ml of 0.1N HCl.

In at least one embodiment of the present disclosure, the speed of the paddle method may be 70 rpm to 150 rpm, such as, but not limited to 80 rpm to 120 rpm, 80 rpm to 150 rpm, or 90 rpm to 120 rpm. In some embodiments, the speed may be 70 rpm, 80 rpm, 90 rpm, 100 rpm, 110 rpm, 120 rpm, 130 rpm, 140 rpm, or 150 rpm, but the present disclosure is not limited thereto.

In at least one embodiment of the present disclosure, the dissolution medium may be of 700 ml to 1000 ml of 0.1N HCl, such as, but not limited to 700 ml to 950 ml, 800 ml to 950 ml, or 850 ml to 1000 ml of 0.1N HCl. In some embodiments, the dissolution medium may be of 700 ml, 750 ml, 800 ml, 850 ml, 900 ml, 950 ml, or 1000 ml, but the present disclosure is not limited thereto.

In at least one embodiment of the present disclosure, the dissolution rate of the pharmaceutical composition of the present disclosure may be over 75% for 45 minutes, such as, but not limited to over 80%, 85%, 90%, 95%, 96%, 97%, 98, or 99% for 45 minutes. In some embodiments, the dissolution rate of the pharmaceutical composition of the present disclosure may be over 85% for 45 minutes after 1 month, 3 months, or 6 months of storage. In some embodiments, the pharmaceutical composition of the present disclosure exhibits an excellent dissolution rate as compared with the commercially available drugs, such as but not limited to Ofev®, in the accelerated stability test.

In at least one embodiment of the present disclosure, the pharmaceutical composition of the present disclosure and the commercially available drugs, such as but not limited to Ofev®, share similar dissolution profile in the in vitro dissolution test, indicating the pharmaceutical product sameness and performance between the pharmaceutical composition and the commercially available drugs.

In at least one embodiment of the present disclosure, the pharmaceutical composition of the present disclosure and the commercially available drugs, such as but not limited to Ofev®, may be bioequivalent in accordance with the results of the in vivo pharmacokinetics test.

### EXAMPLES

Exemplary embodiments of the present disclosure are further described in the following examples, which should not be construed to limit the scope of the present disclosure.

### Example 1. Preparation of Soft Capsule Containing Nintedanib

First, referring to the composition of the capsule shell in Table 1 and Table 2, prepare and mix Gelatin 160, glycerin, and purified water under 65±5° C. and remove the dissolved gases in the mixture. Next, add pigments, iron oxide red, iron oxide yellow, and titanium dioxide, to the aforementioned mixture for obtaining capsule shell solution.

Second, referring to the composition of the pharmaceutical suspension in Table 1 and Table 2, prepare and mix the respective materials (except Nintedanib esylate) by heating to form a solution. Then, add Nintedanib esylate to the aforementioned solution and mix evenly to form the pharmaceutical suspension.

Last, the soft capsule containing Nintedanib is prepared by packing the pharmaceutical suspension into the capsule shell solution with a softgel encapsulation machine. The final pharmaceutical product of the soft capsule containing Nintedanib is obtained by drying under temperature of 15° C. to 25° C. and relative humidity of 15% to 30%.

TABLE 1
Experimental group 1 of the pharmaceutical composition

| | | |
|---|---|---|
| Materials | Unit dose (mg) | Unit dose percentage (%) |

Pharmaceutical composition containing Nintedanib (150 mg)
Composition of pharmaceutical suspension

| | | |
|---|---|---|
| Nintedanib esylate | 180.60 | 43.00 |
| Medium chain Triglycerides | 182.99 | 43.57 |
| Propylene glycol monolaurate | 50.40 | 12.00 |
| (Lauroglycol ™ 90) | | |
| Caprylocaproyl polyoxyl-8 | 4.20 | 1.00 |
| glycerides (Labrasol ® ALF) | | |
| Lecithin | 1.81 | 0.43 |
| Total | 420 | 100 |

Composition of capsule shell

| | | |
|---|---|---|
| Gelatin 160 | 141.84 | 42.98 |
| Glycerin | 61.43 | 18.62 |
| Titanium Dioxide | 0.39 | 0.12 |
| Iron Oxide red | 1.24 | 0.38 |
| Iron Oxide yellow | 1.86 | 0.56 |
| Purified Water | 123.24 | 37.35 |
| Total | 330 | 100 |

TABLE 2
Experimental group 2 of the pharmaceutical composition

| | | |
|---|---|---|
| Materials | Unit dose (mg) | Unit dose percentage (%) |

Pharmaceutical composition containing Nintedanib (150 mg)
Composition of pharmaceutical suspension

| | | |
|---|---|---|
| Nintedanib esylate | 180.60 | 43.00 |
| Medium chain Triglycerides | 191.10 | 45.50 |
| Lauroyl polyoxyl-32 | 6.30 | 1.50 |
| glycerides (Gelucire ® 44/14) | | |
| Lecithin | 42.00 | 10.0 |
| Total | 420 | 100 |

Composition of capsule shell

| | | |
|---|---|---|
| Gelatin 160 | 141.84 | 42.98 |
| Glycerin | 61.43 | 18.62 |
| Titanium Dioxide | 0.39 | 0.12 |
| Iron Oxide red | 1.24 | 0.38 |
| Iron Oxide yellow | 1.86 | 0.56 |
| Purified Water | 123.24 | 37.35 |
| Total | 330 | 100 |

### Example 2. In Vitro Dissolution Test of Soft Capsule Containing Nintedanib (Pharmaceutical Composition (Experimental Group 1))

The in vitro dissolution test may provide an examination of the rate and extent of the release of the pharmaceutical composition, since absorption of the pharmaceutical composition depends on the dissolved profile of pharmaceutical composition. Furthermore, the in vitro dissolution test may be used to assure quality of pharmaceutical product of the pharmaceutical composition.

The comparative group (i.e., Ofev®) and the pharmaceutical composition (Experimental group 1) are subjected to in vitro dissolution test, and the operation process is as follows:
In vitro dissolution test: paddle method with sinker
Speed: 100 rpm;
Dissolution medium: 900 ml of 0.1N HCl; and
Sampling time: 0, 10, 15, 20, 30, 45, and 60 minutes.

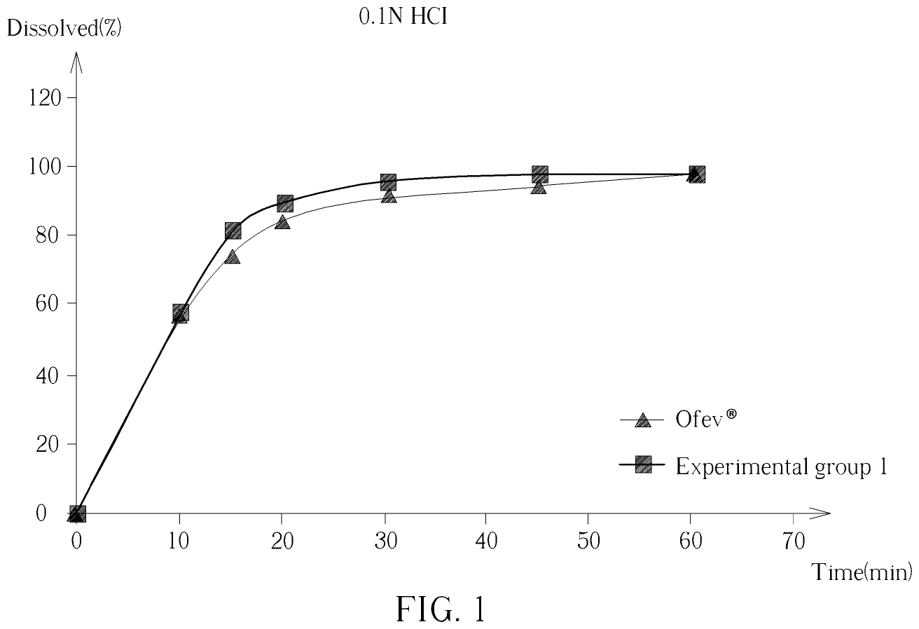

FIG. 1 and Table 3 illustrate the results of the in vitro dissolution test, wherein the comparative group (i.e., Ofev®) and the pharmaceutical composition (Experimental group 1) share similar dissolution profile, indicating an assurance of pharmaceutical product sameness and performance between the comparative group (i.e., Ofev®) and the pharmaceutical composition (Experimental group 1).

TABLE 3
The results of the in vitro dissolution test.

| Sampling time | Comparative group | Pharmaceutical composition |
|---|---|---|
| (min) | (Ofev ®) | (Experimental group 1) |
| 0 | 0.00 | 0.0 |
| 10 | 56.7 | 57.8 |
| 15 | 74.3 | 81.2 |
| 20 | 84.3 | 89.7 |
| 30 | 92.0 | 96.1 |
| 45 | 95.4 | 98.5 |
| 60 | 97.8 | 98.7 |

### Example 3. In Vitro Dissolution Test of Soft Capsule Containing Nintedanib (Pharmaceutical Composition (Experimental Group 2))

The comparative group (i.e., Ofev®) and the pharmaceutical composition (Experimental group 2) are subjected to in vitro dissolution test, and the operation process is as follows:
In vitro dissolution test: paddle method with sinker
Speed: 100 rpm;
Dissolution medium: 900 ml of 0.1N HCl; and
Sampling time: 0, 10, 15, 20, 30, 45, and 60 minutes.

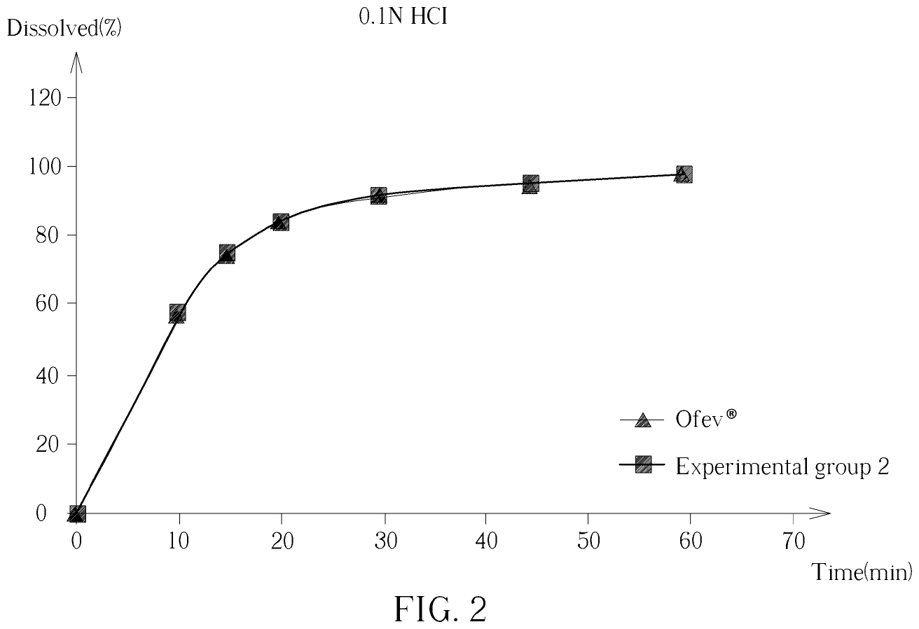

FIG. 2 and Table 4 illustrate the results of the in vitro dissolution test, and the comparative group (i.e., Ofev®) and the pharmaceutical composition (Experimental group 2) share similar dissolution profile, indicating an assurance of pharmaceutical product sameness and performance between the comparative group (i.e., Ofev®) and the pharmaceutical composition (Experimental group 2).

TABLE 4
The results of the in vitro dissolution test.

| Sampling time | Comparative group | Pharmaceutical composition |
|---|---|---|
| (min) | (Ofev ®) | (Experimental group 2) |
| 0 | 0.00 | 0.0 |
| 10 | 56.7 | 56.9 |
| 15 | 74.3 | 75.2 |
| 20 | 84.3 | 84.0 |
| 30 | 92.0 | 91.6 |
| 45 | 95.4 | 96.3 |
| 60 | 97.8 | 97.7 |

### Example 4. Accelerated Stability Test of Soft Capsule Containing Nintedanib (Pharmaceutical Composition (Experimental Group 1 and Experimental Group 2))

The stability testing may provide information on the quality of the pharmaceutical product varies with time under the influence of a variety of environmental factors, e.g., temperature, humidity, and light, and establish a shelf life for the pharmaceutical product and recommended storage conditions. Moreover, in order to increase the rate of physical and performance changes of the pharmaceutical product, an accelerated stability test (40° C./75% RH) instead of a formal stability test is used in Example 4.

Referring to Table 5 to Table 7, evaluation of a change in assay value, dissolution test and impurity test are conducted on the comparative group (Ofev®), the experimental group 1 and the experimental group 2. Testing frequency at the accelerated storage condition includes four time points, including the initial and final time points (e.g., 0, 1, 3, and 6 months). Acceptance criteria of each test is listed in the column “Specification.” Each evaluation item in the accelerated stability test of the experimental group 1 and the experimental group 2 remain within the specification throughout the four time points. However, the dissolution of the comparative group (Ofev®) does not meet the acceptance criteria of over 75% for 45 minutes at time points 1 month and 3 months.

TABLE 5
The results of accelerated stability test of Experimental group 1.

| Item | Specification | 0 month | 1 month | 3 months | 6 months |
|---|---|---|---|---|---|
| Assay | 95.0-105.0% | 100.2% | 101.1% | 99.5% | 102.2% |

Related substance

| | | | | | |
|---|---|---|---|---|---|
| Individual | NMT 0.2% | N.D. | N.R. | N.R. | N.R. |
| Impurity | | | | | |
| Total | NMT 0.5% | 0.0% | 0.0% | 0.0% | 0.0% |
| impurity | | | | | |
| Dissolution | 45 min ≥ 75% | 99% | 98% | 98% | 99% |

NMT: not more than;
N.D.: Not Detected; and
N.R. = Not Reported

TABLE 6
The results of accelerated stability test of Experimental group 2.

| Item | Specification | 0 month | 1 month | 3 months | 6 months |
|---|---|---|---|---|---|
| Assay | 95.0-105.0% | 98.8% | 100.9% | 98.1% | 98.0% |

Related substance

| | | | | | |
|---|---|---|---|---|---|
| Individual | NMT 0.2% | N.R. | N.R. | N.R. | N.R. |
| Impurity | | | | | |
| Total | NMT 0.5% | 0.0% | 0.0% | 0.0% | 0.0% |
| impurity | | | | | |
| Dissolution | 45 min ≥ 75% | 96% | 88% | 90% | 95% |

NMT: not more than;
N.D.: Not Detected; and
N.R. = Not Reported

TABLE 7
The results of accelerated stability test of the
comparative group (i.e., Ofev ®).

| Item | Specification | 0 month | 1 month | 3 months |
|---|---|---|---|---|
| Dissolution | 45 min ≥ 75% | 100% | 74% | 66% |

### Example 5. In Vivo Pharmacokinetics of the Experimental Group 2

The pharmacokinetic parameters are assessed by monitoring variations in concentration of the pharmaceutical product in physiological fluids (e.g., plasma or urines) at different time points after administration, in order to examine the absorption, distribution, biotransformation, and excretion of the pharmaceutical product in the subject. According to the European CPMP and FDA bioequivalence guidance, pharmaceutical products are considered bioequivalent if a 90% confidence interval is contained entirely within 80% to 125%. The pharmacokinetic parameters listed in Table 8 give an indication of the behavior of the pharmaceutical product in the subject's body.

Referring to Table 8, plasma concentrations of 21 human subjects are measured at multiple time points (0.25, 0.5, 1, 1.5, 2, 2.5, 3, 3.5, 4, 4.5, 5, 5.5, 6, 6.5, 7, 8, 10, 12, 24, 48, and 72 hr), after administration of the pharmaceutical composition (i.e., experimental group 2 after meal. For statistical analysis, a 90% confidence interval (CI) is performed. The ratio of experimental group 2/comparative group of each pharmacokinetic parameter falls within the 80% to 125% limit of the European CPMP and FDA bioequivalence guidance with 90% assurance, which demonstrates bioequivalence between the comparative group and the experimental group 2.

TABLE 8
In vivo pharmacokinetic parameters obtained from
plasma concentrations after the administration.

| | Ratio (%) | | |
|---|---|---|---|
| | Experimental group 2/ | Lower limit | Upper limit |
| Dependent | Comparative group | of 90% CI | of 90% CI |
| Cₘₐₓ | 98.90 | 87.38 | 111.93 |
| AUC₀₋ₜ | 101.61 | 92.88 | 111.17 |
| AUC_{0-inf} | 101.70 | 93.03 | 111.18 |

CI = confidence interval
Cₘₐₓ = maximum plasma concentration
AUC₀₋ₜ = area under the curve of a plasma concentration versus time profile (to the last measurable plasma concentration (Cp))
AUC_{0-inf} = area under the curve of a plasma concentration versus time profile (to the last measurable plasma concentration with additional area)

Those skilled in the art will readily observe that numerous modifications and alterations of the embodiments may be made while retaining the teachings of the present disclosure. Accordingly, the above disclosure should be construed as limited only by the metes and bounds of the appended claims.

What is claimed is:

1. A pharmaceutical composition, comprising:
Nintedanib or a pharmaceutically acceptable salt thereof; and
a stabilizer comprising polyoxylglyceride.

2. The pharmaceutical composition of claim 1, wherein the polyoxylglyceride comprises at least one selected from the group consisting of lauroyl polyoxylglyceride, caprylocaproyl polyoxylglyceride, stearoyl polyoxylglyceride, and linoleoyl polyoxylglyceride.

3. The pharmaceutical composition of claim 1, wherein the stabilizer is in an amount of 0.5% to 5% by weight of the pharmaceutical composition.

4. The pharmaceutical composition of claim 1, wherein the stabilizer has a hydrophilic-lipophilic balance value of 11 to 14.

5. The pharmaceutical composition of claim 1, wherein the Nintedanib or the pharmaceutically acceptable salt thereof is in an amount of 20% to 50% by weight of the pharmaceutical composition.

6. The pharmaceutical composition of claim 1, further comprising a carrier and/or a surfactant.

7. The pharmaceutical composition of claim 6, wherein the carrier comprises a lipophilic substance.

8. The pharmaceutical composition of claim 7, wherein the lipophilic substance is at least one selected from the group consisting of vegetable oil, medium chain triglyceride, medium chain partial glyceride, animal derived oil, sorbitan monostearate, ethyl oleate, corn oil glyceride, acetylated monoglyceride, caprylic/capric triglyceride, caprylic/capric/succinic triglyceride, caprylic/capric/linoleic triglyceride, oleic acid, glycerol monoleate, macrogolglycerol caprylocaprate, propylene glycol dicaprylate, and glycerol monolinolate.

9. The pharmaceutical composition of claim 8, wherein the lipophilic substance is the vegetable oil, the medium chain triglyceride, or a combination thereof; and wherein the vegetable oil is soybean oil, coconut oil, castor oil, or a combination thereof.

10. The pharmaceutical composition of claim 6, wherein the carrier is in an amount of 30% to 70% by weight of the pharmaceutical composition.

11. The pharmaceutical composition of claim 6, wherein the carrier has a hydrophilic-lipophilic balance value of 1 to 3.

12. The pharmaceutical composition of claim 6, wherein the surfactant comprises at least one selected from the group consisting of propylene glycol monolaurate, polyglycerol-3 dioleate, propylene glycol monocaprylate, propylene glycol dicaprylate, lecithin, polyethylene glycol sorbitan monolaurate, polyethylene glycol sorbitan monooleate, macrogolglycerol hydroxystearate, macrogolglycerol ricinoleate, macrogolglycerol linoleate, polyglycerol-6 dioleate, and D-a-tocopherol polyethylene glycol succinate.

13. The pharmaceutical composition of claim 6, wherein the surfactant comprises propylene glycol monolaurate, lecithin, or a combination thereof.

14. The pharmaceutical composition of claim 6, wherein the surfactant is in an amount of 0.3% to 25% by weight of the pharmaceutical composition.

15. The pharmaceutical composition of claim 6, wherein the surfactant has a hydrophilic-lipophilic balance value of 3 to 6.

16. The pharmaceutical composition of claim 6, comprising:
the Nintedanib or the pharmaceutically acceptable salt thereof in an amount of 20% to 69.2% by weight of the pharmaceutical composition;
the stabilizer in an amount of 0.5% to 5% by weight of the pharmaceutical composition;
the carrier in an amount of 30% to 55% by weight of the pharmaceutical composition; and
the surfactant in an amount of 0.3% to 20% by weight of the pharmaceutical composition.

17. The pharmaceutical composition of claim 16, wherein
the Nintedanib or the pharmaceutically acceptable salt thereof is in an amount of 20% to 59.2% by weight of the pharmaceutical composition;
the stabilizer is in an amount of 0.5% to 5% by weight of the pharmaceutical composition;
the carrier is in an amount of 40% to 50% by weight of the pharmaceutical composition; and
the surfactant is in an amount of 0.3% to 15% by weight of the pharmaceutical composition.

18. A method for preventing, ameliorating, or treating fibrotic disease and/or lung cancer, comprising administering an effective amount of the pharmaceutical composition of claim 1 to a subject in need thereof.
